(19) United States
(12) Patent Application Publication
(10) Pub. No.: US 2011/0020894 A1
(43) Pub. Date: Jan. 27, 2011

(54) METHOD TO FACILITATE RECYCLING OF BIOMASS DEGRADING ENZYMES

(76) Inventors: ROBERT INGRAHAM, Stillwater, OK (US); ANDREW MORT, Stillwater, OK (US); MARK WILKINS, Stillwater, OK (US)

Correspondence Address: FELLERS SNIDER BLANKENSHIP;BAILEY & TIPPENS, THE KENNEDY BUILDING, 321 SOUTH BOSTON SUITE 800, TULSA, OK, 74103-3318, US

(21) Appl. No.: 12/841,148

(22) Filed: Jul. 21, 2010

Related U.S. Application Data
(60) Provisional application No. 61/227,359, filed on Jul. 21, 2009.

(51) Int. Cl. C12N 11/14

(52) U.S. Cl. 435/176

(57) ABSTRACT

A strategy for enzymatic biomass conversion that involves enzyme recovery and reuse. Enzyme recovery is achieved by attaching enzymes to paramagnetic nanoparticles. By magnetically attracting the paramagnetic nanoparticles, enzymes may be recovered for reuse. The process of enzymatic biomass hydrolysis includes: attaching biomass degrading enzymes to a surface of functionalized particles having paramagnetic properties; mixing the particles with a biomass; recovering the enzymes via magnetic separation; and re-using the enzymes in a subsequent process. The process provides a product feed stream which may be subjected to further processing. The process may be repeated multiple times wherein the biomass is sequentially subjected to different enzymes.

### CROSS REFERENCE TO RELATED APPLICATIONS

This application claims the priority of U.S. Provisional Patent Application No. 61/227,359 entitled “METHOD TO FACILITATE RECYCLING BIOMASS DEGRADING ENZYMES,” filed Jul. 21, 2009, the contents of which are hereby incorporated by reference.

### STATEMENT REGARDING FEDERALLY SPONSORED RESEARCH OR DEVELOPMENT

This invention was made with government support under grant number DE-FG02-96ER20215 awarded by the Department of Energy. The government has certain rights in the invention.

### FIELD OF THE INVENTION

The present invention relates generally to methods of biomass hydrolysis and more particularly to recovery of enzymes.

### BACKGROUND OF THE INVENTION

Ethanol may be derived from biomass. Fermentable sugars, glucose and xylose are produced from hydrolysis of cellulose and hemicellulose. Sugars are fermented by microorganisms, such as bacteria or yeast, to produce ethanol.

Approximately 15 to 25% of the biomass in the biosystem is comprised of lignin. Lignin is a complex network of aromatic compounds having a high energy content. Approximately 23% to 32% of the biosystem is comprised of hemicellulose. Hemicellulose is a collection of unusual five and six carbon sugars linked together in long chains. Xylose is the second most abundant sugar in the biosphere. The remaining 38% to 57% of the biomass in the biosphere is comprised primarily of cellulose. Cellulose is comprised of long chains of glucose and is the most abundant form of carbon in the biosphere.

Conversion of biomass to energy has traditionally run into one of several problems. The first problem relates to enzymatic access to biomass polymers. Enzymatic access to cellulose is hindered by a complex and interwoven biopolymer matrix found in plant cell walls. As a result, complete enzymatic hydrolysis of cellulose is virtually unattainable.

The second problem relates to the formation of incompatible by-products that may be encountered during chemical or enzymatic hydrolysis. In enzymatic hydrolysis, lignin degradation produces phenolic compounds that are toxic to cellulase and xylanase enzymes. These enzymes produce fermentable sugars for ethanol production.

Current strategies for biomass conversion include chemical biomass hydrolysis. Acid hydrolysis is the most complete method of biomass digestion available. Nevertheless, the method is destructive and degrades a large portion of the fermentable sugars. Alkaline hydrolysis is less damaging to the produced sugars. However, with this method biomass hydrolysis is far from complete. Additionally, by-products from these methods interfere with the subsequent microbial fermentation process.

Other strategies currently employed for biomass conversion include enzymatic biomass hydrolysis. This method is not destructive and can produce a substantial amount of fermentable sugars. Use of specific enzymes reduces or eliminates interfering by-products. For example, cellulase and xylanase enzymes will digest biomass polymers cellulose and hemicellulose to fermentable sugars without releasing harmful by-products.

However, due to the complex and interlocking polysaccharide matrix that forms plant cell wall structure, the development of an economically sustainable robust hydrolysis method for the conversion of lignocellulosic biomass to ethanol has been unattainable with conventional methods.

Enzymatic hydrolysis, however, is cost prohibitive in either of the two methodologies currently employed. In the first, i.e., batch fermentation, expensive biomass degrading enzymes are discarded with the spent media. In a second methodology, i.e., in a continuous feed bioreactor, expensive biomass degrading enzymes are lost in the effluent.

Additional problems encountered with enzymatic hydrolysis include that of incomplete biomass digestion. The biopolymer lignin interferes with total cellulose and hemicellulose digestion. Further, enzymes that degrade lignin produce phenolic compounds that are toxic to the cellulase and xylanase enzymes that produce fermentable sugars for ethanol production.

The economics of enzymatic hydrolysis of biomass into fermentable sugars are further pressured by the high cost of biomass degrading enzymes. Moreover, the development of a robust enzymatic hydrolysis recovery/reuse method to convert lignocellulosic biomass to ethanol that is economically sustainable has been greatly hampered by the complex and interlocking polysaccharide matrix which forms plant cell wall structure.

We expect the conversion of post-harvest agricultural waste to biofuels to become a profitable business reality and a value-added process for those in the agribusiness sector. Increasing the economic feasibility of biofuels production could significantly reduce our country's dependency on importing foreign oil. Since ethanol facilities are most likely to be constructed in rural areas near the feedstock, ethanol facilities will provide new jobs and an influx of additional income to state rural economies. In addition, lost crops and resulting profits from recent climactic changes could be partially recovered through this improvement in biomass conversion technology.

### SUMMARY OF THE INVENTION

Recent improvements in enzyme production, cost, and availability still prohibit achieving economic sustainability and widespread commercialization and use of cellulosic biofuels and biobased products. There is nothing efficient about losing expensive biomass degrading enzymes when they can easily be recovered and reused. The invention of the present disclosure will advance the science of biofuels and biobased goods by introducing a robust enzyme recycling method. The invention uses enzyme coated microbeads with magnetic properties and magnetic separation techniques to attain the goal of enzyme recovery and reuse.

One objective of the invention is to advance the progress and efficiency of enzymatic biomass hydrolysis. This objective will be accomplished through the recycling of biomass degrading enzymes by attaching the enzymes to surface functionalized microbeads with magnetic properties, thereby allowing the collection and reuse of expensive and viable enzymes. In addition, the enzyme coated microbeads can selectively target the hydrolysis of the biomass polymers such as hemicellulose (xylan) which will free a larger quantity of cellulose for hydrolysis to glucose and conversion to biofuels in subsequent or simultaneous fermentation.

Currently, the hydrolysis of biomass polysaccharides into fermentable sugars is accomplished by chemical or enzymatic means. Chemical hydrolysis of lignocellulosic biomass, while being less expensive than enzymatic hydrolysis, tends to produce by-products furfural and HMF that are toxic to ethanol producing organisms such as S. cerevisiae. Also, due to harsh reaction conditions, chemical hydrolysis and fermentation are not accomplished simultaneously.

Enzymatic hydrolysis provides the best yield of undamaged fermentable sugars with few interfering by-products. However, due to their physical properties, enzymes cannot be easily recovered for reuse in subsequent hydrolysis reactions. Enzyme loss is also an issue in combined enzymatic hydrolysis and fermentation reactions. For example, in continuous-feed bioreactors that are used for Simultaneous Saccharification and Fermentation (SSF), the average time enzymes are retained is limited to the reactor's hydraulic retention time (HRT) rather than to the enzyme's functional lifetime. Typically the functional life of hydrolysis enzymes is significantly longer than a bioreactor's HRT. The net result is the unrecoverable loss of active enzymes through the reactor's effluent stream. Even with recent improvements enzyme replacement in biomass hydrolysis remains economically unviable for commercial applications.

Another difficulty in lignocellulose conversion to ethanol is the inability to access the cellulose within the lignocellulosic cell wall matrix. Due to the nature of this invention, i.e., the ability to remove enzymes from reaction mixtures, cellulose fibers can now be further exposed by alternating, repeated and selective erosion of its surrounding biopolymer cage. Individual groups of enzymes on coated microbeads that target the specific biopolymers can now be added to or collected from hydrolysis reactor mixtures. The select enzymes are added (e.g., endo- and exo-xylanase mix) and the biopolymer hemicellulose (xylan) is digested. The digestion mixture, now containing the fermentable sugar xylose is flushed from the reaction chamber. As the chamber flushed, the xylanase enzymes are magnetically withdrawn from the exit line effluent stream as the reactor chamber is flushed with fresh hydrolysis buffer. Now, selective digestion of the newly exposed cellulase fibers can begin, or other restrictive biopolymers lignin or pectin can be digested and their once interfering by-products removed.

With the ability to selectively target hemicellulose, or lignin or pectin in individual reactions, useful and/or toxic by-products from biopolymer digestion can now be flushed from the hydrolysis mixture (see FIG. 5, regarding the concept of selective/sequential biopolymer digestion into separate feed streams). Through this process, biomass polymer products can be funneled into separate and useful feed streams to produce value added bio-pharmaceuticals and other bio-based products. In addition, through this process, cellulose fibers will be made more accessible to cellulose degrading enzymes leading to a significantly greater biomass to ethanol yield. This approach will greatly increase the amount of available cellulose while also producing large amounts of xylose, resulting in a net increase of total fermentable sugars, thereby significantly reducing the cost of bio-ethanol production.

A second objective is an innovative method for recovering biomass degrading enzymes from hydrolysis reactors or retaining enzymes in SSF bioreactors by immobilizing enzymes on the surface of magnetic microscale beads. By attaching lignocellulose hydrolyzing enzymes to magnetic microscale beads, we can utilize magnetic separation technologies to divert and return enzymes from the fermentation culture effluent stream back to the bioreactor chamber. Enzymes can also be magnetically collected from hydrolysis pretreatments and reused in subsequent hydrolysis pretreatment reactions.

Additional objectives of the invention include covalently linking biomass degrading enzymes with surface-functionalized magnetic-microbeads and assay enzyme coated microbeads for post-attachment hydrolysis activity.

Enzymes that hydrolyze biomass polymer (polysaccharides) can be covalently coupled with paramagnetic silica beads and maintain their enzymatic activity. Once attached, these enzymes are tethered to the bead's surface by an extended hydrocarbon chain (≈30 atoms long), which allows the enzyme freedom of movement, spatial orientation in solution, and retention of enzymatic activity. Exposed amino acids on the enzyme's surface (i.e., primary amines, hydroxyl or sulfhydryl groups) are covalently coupled to epoxide groups on the functionalized surface of microbeads. To test/prove this attachment and enzyme recovery process an “endo-beta-1,4-xylanase” for experimental testing. Approximately 40% to 60% total bead surface coverage was achieved. The functionalized microbead surface was coated with an average of approximately 100,000 xylanase enzymes per microbead. These xylanase coated microbeads retained their enzymatic activity (FIGS. 2-4).

A further objective includes the recovery of enzymes from a hydrolysis reactor through magnetic separation and test enzymatic activity retention through multiple cycles in subsequent reactions.

Enzyme coated microbeads can be recovered and reused in subsequent agricultural crop waste hydrolysis reactions. During hydrolysis experiments, cellulose fibers were exposed by selectively targeting xylan, a structural polysaccharide that is the primary component (backbone) of hemicellulose. Lignin and pectin surround and bind to hemicellulose, the polymer which tightly binds to and surrounds cellulose fibers. A substantial increase in the amount of available cellulose for subsequent or simultaneous cellulase digestion such as that carried out in an SSF reaction is expected. This process is believed to produce large amounts of xylose resulting in a significant increase of total fermentable sugars. Examples of hydrolysis substrates include corn and/or sorghum stover.

In summary, the present invention proposes a new strategy for enzymatic biomass conversion that involves enzyme recovery and reuse. Enzyme recovery is achieved by attaching enzymes to paramagnetic nanoparticles. By magnetically attracting the paramagnetic nanoparticles, enzymes may be recovered for reuse.

The present invention is a method for recovering enzymes from hydrolysis batch reactors or retaining enzymes in SSF continuous flow bioreactors. Using the method of the invention, lignocellulosic hydrolyzing enzymes can be diverted from fermentation culture effluent streams and returned to the bioreactor chamber. Enzymes can also be collected from SSF hydrolysis pretreatments and reused in subsequent hydrolysis pretreatment reactions.

The present disclosure describes a method of recovering enzymes from enzymatic biomass hydrolysis. The process includes, in a basic embodiment, the steps of obtaining functionalized particles having paramagnetic properties; attaching biomass degrading enzymes to the functionalized particles; mixing the particles having attached enzymes with a biomass substrate to form a mixture; and recovering the particles with attached enzymes from the mixture via magnetic separation. The process provides a product feed stream which may be subjected to further processing. For example, a glucose product feed stream resulting from subjection of cellulose with cellulase might be transported to a fermentation reactor to produce saleable products such as ethanol. The process may be repeated multiple times wherein the biomass in the reactor is sequentially subjected to different enzymes to produce multiple product feed streams.

### BRIEF DESCRIPTION OF THE DRAWINGS

FIGS. 1A-1D show an exemplar xylanase recovery process.

FIG. 2 graphically demonstrates the successful attachment of xylanase enzyme from solution and retention of enzymatic activity.

FIG. 3 graphically confirms that xylan degradation activity is due to successful enzyme attachment to microbeads and is not just an artifact of the microbeads.

FIG. 4 graphically demonstrates enzyme recovery and reuse.

FIG. 5 is a schematic representation of a bioreactor capable of sequential hydrolysis reaction processes employing multiple enzymes and capable of recovery and reuse of enzyme coated microbeads according to the present disclosure.

FIG. 6 depicts an exemplary bioreactor capable of performing the processes of FIG. 5.

FIG. 7 depicts the chemical formulae for various functionalized particles.

### DETAILED DESCRIPTION OF THE PREFERRED EMBODIMENTS

The present invention utilizes the attachment of an enzyme to a functionalized paramagnetic particle, such as a glass-iron composite spherical bead or “microbead”. The enzyme is used to degrade biomass, many of which are well known in the art at present, such as but not limited to: xylanase, cellulose, lacasse, ligininase, pectin-lyase and/or pectinase. Since the microbead has paramagnetic properties, it can be collected, along with the attached enzyme, for reuse in subsequent biomass digestion reactions.

Example microbeads may be obtained from Bioclone Inc., 7965 Silverton Ave., Suite 1309, San Diego, Calif. 72126 and include but are not limited to the following: Amine-terminated beads; DADPA-terminated beads; Carboxy-terminated beads; Carboxy-terminated beads; Epoxy-activated beads; Expoy-activated beads; Aldehyde-modified beads; Aldehyde-modified beads; Hydrazide-modified beads; IDA-modified beads; Silica-modified beads.

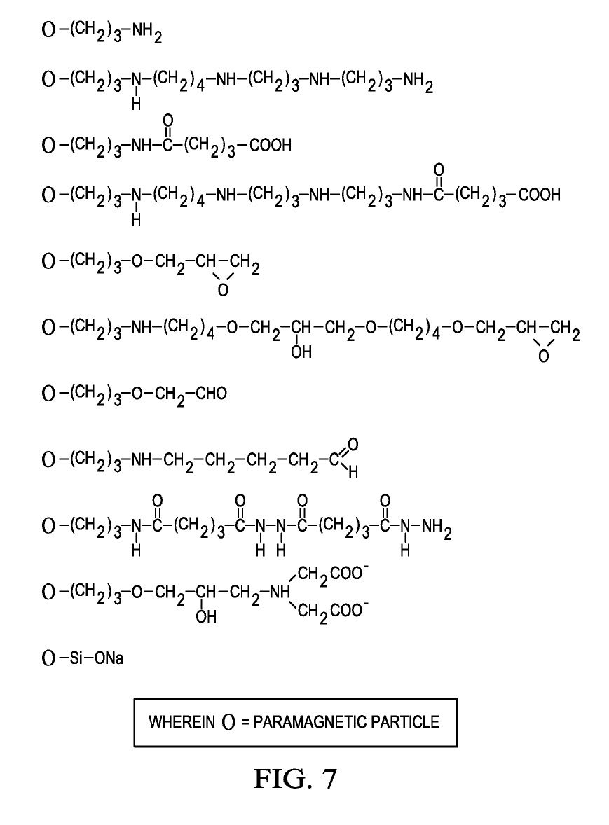

The example microbeads and functional groups of the above list, are depicted in FIG. 7.

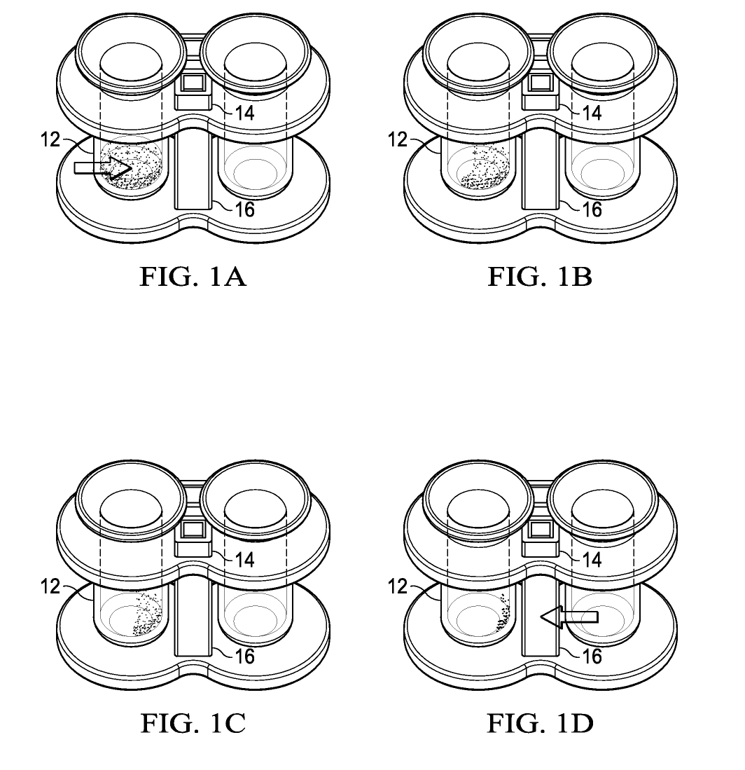

FIGS. 1A-1D show microbeads with attached enzyme being pulled from reaction buffer during enzyme recovery experiments. FIGS. 1A-1D show an exemplar xylanase recovery process. In this example, a xylanase enzyme is used which hydrolyzes the biomass polymer hemicellulose. After the first reaction (run 1), the reaction mixture containing xylanase coated microbeads (XCMs) was collected in a micro-centrifuge tube and placed in a magnetic holder 14 (left tube 12, FIG. 1A). As shown in FIGS. 1A-1D, the paramagnetic microbeads with attached xylanase in solution were separated from solution over a period of two minutes (see, FIGS. 1B-1D). As can be seen in FIG. 1D, after approximately two minutes has elapsed, the paramagnetic microbeads gravitate towards a magnet housed in pillar 16 of magnetic holder 14, as indicated by the arrow of FIG. 1D. The result is a reaction buffer having a substantially clear appearance of FIG. 1D as compared to the cloudy appearance of the reaction buffer of FIG. 1A. The spent supernatant (reaction mixture minus the paramagnetic microbeads with attached xylanase) from the first digestion experiment is withdrawn (left tube 12) and discarded while the XCMs remain in the tube. In an industrial process, the supernatant would be the product feed-stream, which would be transported for further processing, e.g, for fermentation. The XCMs are immediately resuspended and “washed” with buffer solution three times to remove digested material from previous reaction. XCMs (described below) are then resuspended in reaction buffer immediately, in preparation for next biomass degradation experiment.

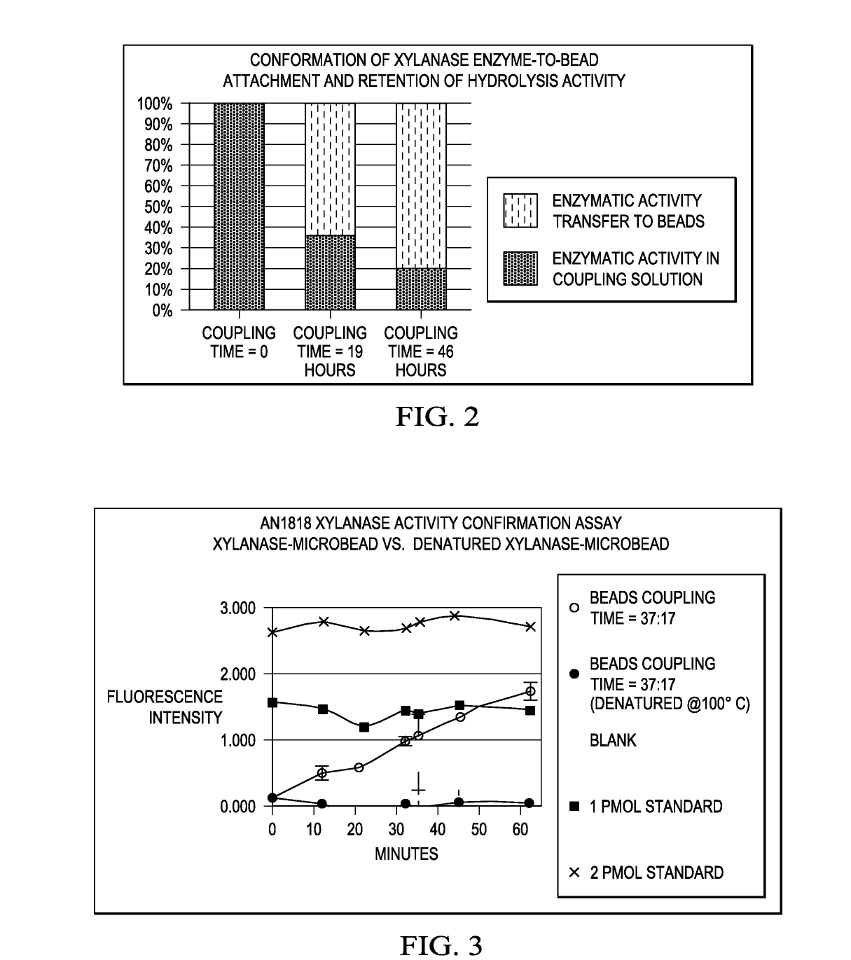

Three sets of experiments were conducted to test attachment, activity and recoverability of a hemicellulose degrading enzyme endo-xylanase (AN1818.2) (FIGS. 2 and 3). This enzyme is a family 10 xylanase (NCIB website, http://www.ncbi.nlm.nih.gov/protein) known to have both xylanase and cellulose degrading ability. In all experiments, the enzyme was covalently attached to approximately 1 μm paramagnetic beads using Bioclone's attachment protocol. The attachment procedure is set forth below. Activity assays used larch wood xylan substrate or the Molecular Probes, Inc. EnzChek® Ultra Xylanase Assay Kit (E33650) purchased from Invitrogen. Experimental results demonstrate approximately 40% to 60% coverage of the functionalized microbead surface with an average of approximately 100,000 xylanase enzymes per microbead.

Successful attachment of the xylanase enzyme from solution and retention of enzymatic activity is demonstrated in FIG. 2. Confirmation that xylan degradation activity is due to successful enzyme attachment to microbeads and is not just an artiface of the microbeads is shown in FIG. 3. This test was conducted with active vs. heat denatured enzyme coated microbeads.

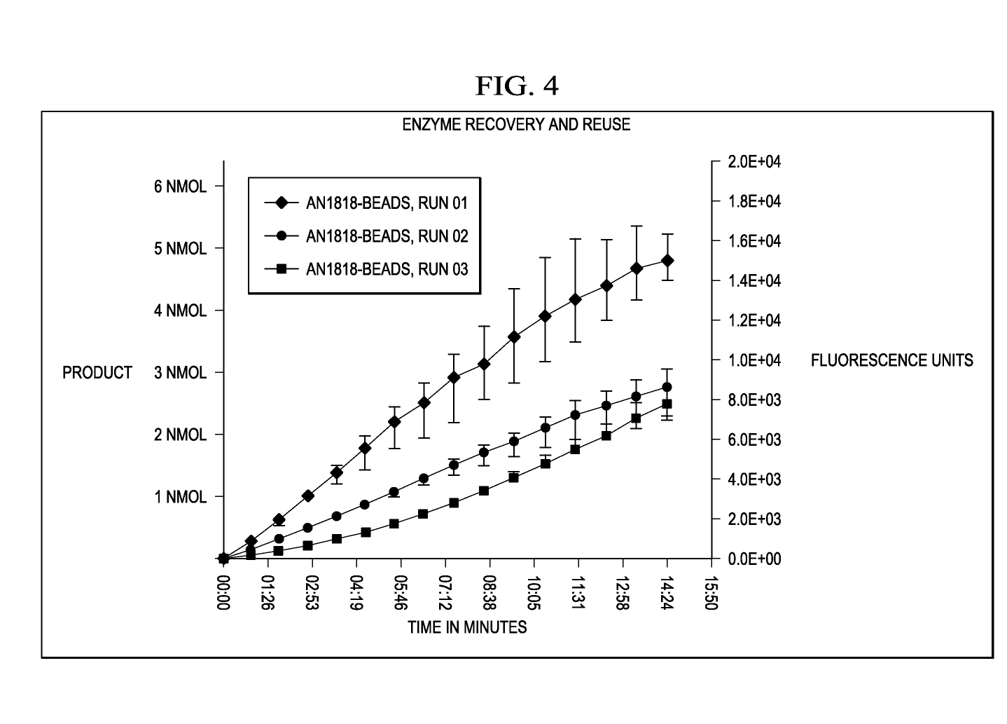

Enzyme recovery and reuse are demonstrated in FIG. 4, run 1, run 2, and run 3 were accomplished with the same XCMs. FIGS. 1A-1D illustrate the recovery protocol. Retention of XCMs in the micro-centrifuge tube by the magnetic holder was not perfect and there was some loss of XCMs during washing steps, however, runs 2 and 3 show excellent repeatability and enzyme recoverability.

Bead binding efficiency of purified AN 1818.2 vs. unpurified AN 1818.2 from production culture medium was tested. The most enzyme activity to microbead attachment was obtained using the purified enzyme with a concentration of 2.6 μg of enzyme/mL vs. the unpurified enzyme with concentration of 430 μg of protein/mL.

An example list of nanoparticle functionalized attachment groups that may be used for covalent enzyme attachment include but are not limited to epoxide, carboxy, amine, aldehyde, DADPA, or hydrazide. It is understood that other nanoparticle functionalized attachment groups may be apparent to one skilled in the art.

Experimental procedures for meeting the below enumerated objectives are described below.

Objective 1: Biomass degrading enzymes are covalently linked with surface-functionalized paramagnetic-microbeads and assay enzyme coated microbeads for post-attachment hydrolysis activity. Temperature should be optimized and pH hydrolysis conditions for xylanase coated microbeads. Temperature should be optimized and pH hydrolysis conditions should be optimized for cellulase coated microbeads. A range of sizes of microbeads from 50 nm-1 μm are contemplated with three sizes, 50 nm, 100 nm, and 1 μm preferred. The surface of paramagnetic silica microbeads is treated with chemically active functional groups that can covalently bind enzymes with xylanase and cellulase activities thus producing enzyme coated microbeads. Using their magnetic properties, the enzyme coated microbeads may be recovered and reused in subsequent biomass hydrolysis reactions.

Enzyme Selection

To enhance the biomass to ethanol yield, endo xylanase is used to selectively target xylan, which is the primary component of hemicellulose that interlinks cellulose fibers and which tightly binds cellulose to other structural polysaccharides such as lignin and pectin. This approach is calculated to substantially increase the amount of available cellulose by exposing cellulose fibers for subsequent cellulase digestion to glucose. This process will also generate high amounts of xylose resulting in a significant increase in the amount of total fermentable sugars which will ultimately lead to a higher yield of ethanol.

Research into the structure and synthesis of plant cell walls combined with the recent intense interest in lignocellulosic biomass conversion to biofuels and other products has created the need for easy access to a range of biomass degrading enzymes.

Paramagnetic microbeads may be purchased from Bioclone Inc. 7965 Silverton Ave. Suite 1309, San Diego, Calif. 92126 or other suitable supplier known in the art.

The protocol for covalent attachment of biomass degrading enzymes such as xylanase, cellulose, lacasse, lignose, pectin-lyase or pectinase to epoxide groups on microbead surface is set forth by Bioclone Inc. (http://www.bioclon.com/functional-magnetic-beads-epoxy.html). The protocol is provided below.

Procedures

Use Centriprep Ultacel YM-10, 10 kDa Cat. No. 4321, follow the manufacturer's instructions (Centriprep) protocol (used for exchanging endo-beta-1,4-xylanase into coupling buffer). Centriprep YM-10, 10 kDa Cat. No. 4321 is a product of Millipore, Inc. 290 Concord Road, Billerica, Mass. 01821. Prepare coupling buffer; follow the manufacturer's instructions, i.e. Bioclone Inc. (http://www.bioclon.com/functional-magnetic-beads-epoxy.html).

Produce enzyme coated microbeads using endo-beta-1,4-xylanase, e.g., xylanase coated microbeads using xylanase coupled with epoxide coated paramagnetic beads (1 μm diameter), and determine enzymatic hydrolysis activity units under standard conditions.

Exchange enzyme in solution into coupling buffer using Centriprep®; see, e.g, Bioclone Inc. Protocol for coupling of enzyme to microbeads

Buffers

Ionic coupling buffer strengths are crucial to obtain the high coupling efficiency rate.

Coupling buffer should be at minimal ionic strength

Buffer does not contain any amino (e.g. Tris) or other nucleophiles
1. Coupling Buffer: 0.1 M sodium phosphate, pH 8.0
   8.62 g of sodium phosphate dibasic
   5.42 of sodium phosphate monobasic
   800 mL ddH2O
      Adjust the final pH to 8.0 with 0.1 N HCl
      Adjust the final volume to 1 L with ddH2O
2. Storage Buffer: Phosphate Buffer saline (PBS), pH 7.4
0.02% NaN3
1.82 g/L Potassium phosphate dibasic
0.22 g/L Sodium phosphate monobasic
8.76 g/L Sodium chloride
NaN3 0.2 g/L
   Adjust the final pH to 7.4 with 1 N HCl or NaOH
   Bring to final volume of 1 L with ddH2O

Exchange Enzyme in Solution to Coupling Buffer

Modified (Centriprep Protocol used for exchanging endo-beta-1,4-xylanase into coupling buffer). The Centriprep protocol is available from Millipore, Inc., 290 Concord Road, Billerica, Mass. 01821.

1. Open Centriprep tube
Turn twist-lock cap counterclockwise on sample container
   Do not remove completely
Slide filtrate collector assembly out, set aside
   Do not touch, scratch or damage the membrane

2. Add solution
Add 1 mL of enzyme solution with an activity≈40 U per mL
   Add coupling buffer to fill line of sample container
   15 mL Maximum total volume

3. Seat twist-lock cap fully onto shoulder of filtrate collector
Slide cap downward until it stops at shoulder

4. Slide the capped filtrate collector into sample container
Collector displaces solution
Turn twist-lock cap clockwise to seal the sample container
   Be sure air-seal cap is tight on twist-lock cap

5. Insert assembled Centriprep apparatus into 50 mL opening in centrifuge bucket insert
Counter balance with water filled 50 mL tube

6. Centrifuge at 4° C. and 4,000 rpm using Sorvall Legend RT Plus centrifuge with swinging-bucket rotor
Centrifuge until fluid levels equilibrate
Snap off the air-seal cap
Decant the filtrate
Replace cap and spin device again

7. Centrifuge at 4° C. and 4,000 rpm using Sorvall Legend RT Plus centrifuge with swinging-bucket rotor
Centrifuge until fluid levels equilibrate
Snap off the air-seal cap
Decant the filtrate
Add coupling buffer to the fill line
Replace cap and spin device again

8. Repeat steps 6 and 7 two times

9. Decant the remaining filtrate

10. Centrifuge at 4° C. and 4,000 rpm using Sorvall Legend RT Plus centrifuge with swinging-bucket rotor
Centrifuge again and decant filtrate
Repeat until the retentate volume=1 mL
Snap off the air-seal cap
Decant the filtrate

11. Prepare enzyme for further use or storage
Turn twist-lock cap counterclockwise to loosen.
Take out filtrate collector
Use a pipette to transfer the enzyme in coupling buffer solution (retentate) to a 2 mL micro-centrifuge tube
Adjust solution volume to 1 mL to match the volume of starting solution to retain original enzymatic activity level
   Assay enzyme solution to verify
Place tube in ice bath or refrigerate at 4° C. for immediate use in enzyme coupling reaction with functionalized paramagnetic microbeads

Couple enzyme to BcMag Epoxy-modified Magnetic Beads

(Bioclone Inc. Http://Www.Bioclon.Com/Functional-Magnetic-Beads-Epoxy.Html)

1. Transfer 1 mL (10 mg) of completely suspended paramagnetic microbeads to a 2 mL micro-centrifuge tube
Place tube into magnetic separator, (see FIG. 1a)
Wait until the supernatant is clear, (see FIG. 1d)
Carefully remove the supernatant so as to not disturb the sequestered microbeads
Discard supernatant

2. Wash the beads
Remove tube from magnetic separator
Add1 mL of coupling buffer
Resuspend beads by shaking tube vigorously
Place tube into magnetic separator (see FIG. 1a)
Wait until the supernatant is clear (see FIG. 1d)
Carefully remove the supernatant so as to not disturb the sequestered microbeads
Discard supernatant

3. Repeat step 2 three times

Note: It is preferred to not let the beads dry out between washing steps or enzyme transfer.

4. For optimal attachment of endo-beta-1,4-xylanase to epoxy functionalized microbeads
Dilute enzyme to a working concentration of 25 μg/mL using coupling buffer
   e.g., If the starting enzyme activity=40 Upper mL, the starting enzyme concentration=430 μg/mL
   Use 58 μL of the starting enzyme solution (from step 9 of the previous Centriprep Protocol) and 942 μL of coupling buffer to achieve the 25 μg/mL working concentration

5. Add xylanase to washed microbeads for coupling reaction
Add 1 mL of enzyme (working concentration) to the washed beads from step 3
   Note: It is preferred to not let beads dry out during enzyme transfer.
Remove tube from magnetic separator
Resuspend beads and mix thoroughly

6. Place reaction tube in holder on orbital shaker
Shake at 300 rpm at room temperature for 48 hours

7. Wash beads
Wash beads 10 times using 1 mL of 1 M NaCl as in step 2

8. Resuspend enzyme coated microbeads in 1 mL of storage buffer

9. Store at 4° C.

(Coupling protocol obtained from Bioclone Inc. http://www.bioclon.com/) and modified as needed

Establish Xylanase Post Attachment Activity Retention

Establish enzymatic hydrolysis activity of the endo-beta-1,4-xylanase coated microbeads using the Molecular Probes® EnzChek® Ultra Xylanase Assay Kit from Invitrogen. Hydrolysis samples are analyzed in triplicate using a 96 well plate format with a Turner BioSystems Modulus™ II Microplate Fluorescence Reader. The Turner BioSystems Modulus™ II Microplate Fluorescence Reader is a high throughput system. In a single day at one run per hour, up to 160 hydrolysis reaction conditions can best tested while allocating 36 wells per 96 well plate run to be used for assay controls and standards. Experimental controls including blanks, reaction buffer blanks, microbead blanks and denatured enzyme coated microbeads etc. will be used to validate experimental results. Accuracy of fluorescence assays will be confirmed using a traditional 2-cyanoacetamide assay or similar established method (Bauer et al. 2006).

Assay endo-beta-1,4-xylanase coated microbeads for xylan and cellulose hydrolyses activity and determine optimal hydrolysis pH and temperature (Note: A family 10 xylanase should also have cellulase activity).

Establish the xylan hydrolysis activity of the xylanase coated microbeads using the Molecular Probes® EnzChek® Xylanase Assay Kit from Invitrogen, Inc., 5791 Van Allen Way, Carlsbad, Calif. 92008.

Establish the cellulose hydrolysis activity of the xylanase coated microbeads using the Molecular Probes® EnzChek® cellulase substrate *blue fluorescent, 339/452 assay kit from Invitrogen.

Hydrolysis samples are analyzed in triplicate using a 96 well plate format with a Turner BioSystems Modulus™ II Microplate Fluorescence Reader as stated previously in Section 1.

Produce endo-beta-1,4-xylanase coated microbeads using xylanase and epoxide coated magnetic beads of various sizes (i.e. 50 nm, 100 nm and 500 nm diameters). A preferred xylanase is endo-beta-1, 4-xylanase; however, it is understood that other suitable xylanase is commercially available. Determine enzymatic activity units under standard conditions. Compare results with previous data from approximately 1 μm diameter enzyme coated microbeads to determine optimum microbead size.

Produce xylanase coated microbeads of selected sizes. Follow coupling procedures as detailed in Objective 1, Section 1. Establish the xylan hydrolysis activity of the xylanase coated microbeads using the Molecular Probes® EnzChek® Ultra Xylanase Assay Kit from Invitrogen.

If applicable (depending on to outcome of Section 2), establish the cellulose hydrolysis activity of the xylanase coated microbeads using the Molecular Probes®, EnzChek® cellulose substrate *blue fluorescent, 339/452 assay kit from Invitrogen.

Hydrolysis samples are analyzed in triplicate using a 96 well plate format with a Turner BioSystems Modulus™ II Microplate Fluorescence Reader as stated previously in Section 1.

Produce cellulose degrading enzyme coated microbeads using an endo-cellulase enzyme coupled to epoxide coated magnetic beads of various sizes (i.e. 50 nm, 100 nm, 500 nm and 1 μm diameter beads). Determine enzymatic activity units under standard conditions. Determine optimum pH, temperature and microbead size for best activity retention and enzyme durability.

Produce cellulose coated microbeads of selected sizes. Follow coupling procedures as detailed in Section 1 of Objective 1.

Establish the cellulose hydrolysis activity of the cellulase coated microbeads using the Molecular Probes® EnzChek® cellulase substrate *blue fluorescent, 339/452 assay kit from Invitrogen.

Hydrolysis samples are analyzed in triplicate using a 96 well plate format with a Turner BioSystems Modulus™ II Microplate Fluorescence Reader as stated previously above.

Based preliminary studies and published data, we expect the xylanase and cellulase enzymes to remain active after magnetic microbead attachment resulting in increased hydrolysis of hemicellulose and cellulose. Due to our previous studies involving the successful recovery and reuse of enzyme coated microspheres which retained enzymatic activity during three hydrolysis reactions, as well as substantiating studies found in literature, we again expect successful activity retention after bead coupling and through successive recovery and reuse experiments.

Functionalized nano/microbeads with less aggressive functionalized attachment groups such as amino, DADPA, Carboxy, aldehyde and hydrazide surface groups may be used in lieu of a functionalized epoxide bead surface. Attachment of active enzymes with the former chemical groups will result in less covalent bonds between the enzyme and bead surface. This could be advantageous if the epoxide to enzyme binding interferes with the bound enzyme's active or regulatory sites.

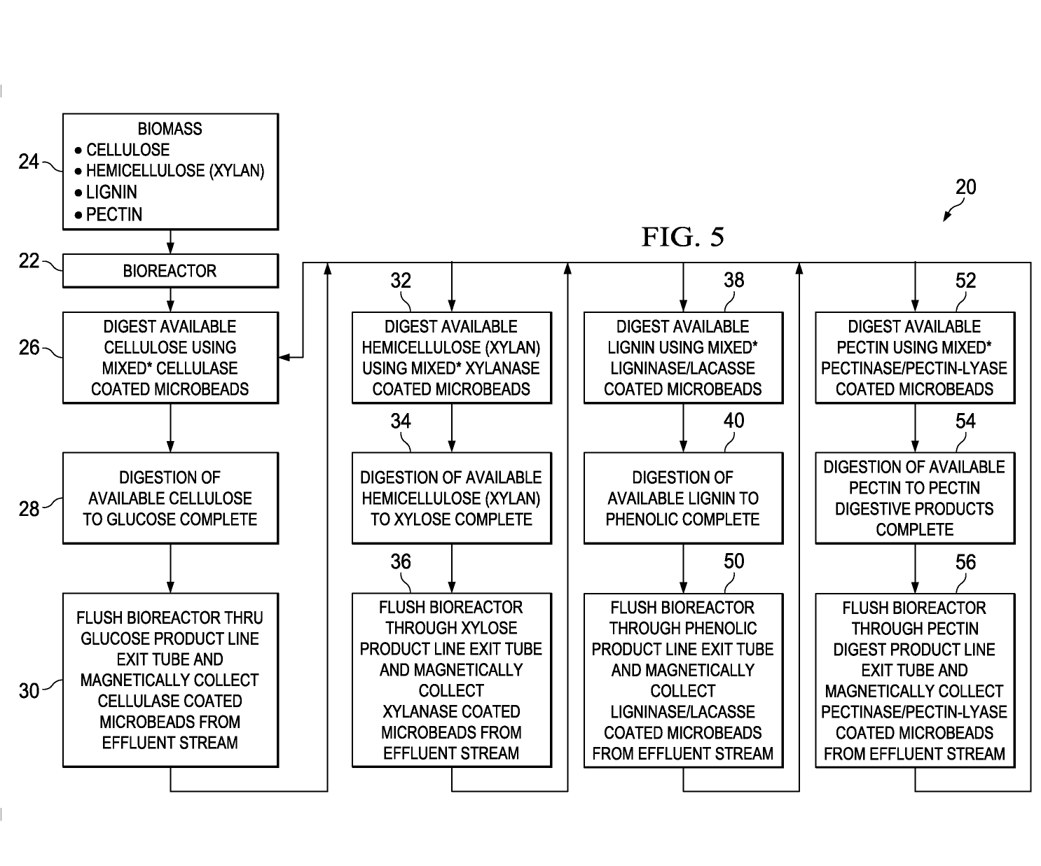

Referring next to FIG. 5, which depicts a schematic representation of a bioreactor capable of sequential hydrolysis reaction process employing multiple enzymes. The bioreactor is capable of recovery, regeneration, and reuse of the reactive enzymes by employing coded paramagnetic microbeads as described above. Parameters for operating enzyme hydrolysis bioreactors are generally known.

With reference to FIG. 5, a sequential enzyme hydrolysis process and system 20 includes a bioreactor 22 into which a selected biomass is deposited for selective enzyme hydrolysis. The system 20 is capable of carrying out multiple sequential enzyme biomass hydrolysis reactions in order to produce multiple product feed streams from the selected volume of biomass. Each sequential enzyme hydrolysis reaction is conducted using an enzyme which is attached to the surface of paramagnetic microbeads and the process carried out as described above so that each selected enzyme may be recovered and reused until they no longer function.

In the embodiment depicted in FIG. 5, a biomass is selected at 24, which includes cellulose, hemicellulose (Xylan), lignin, and pectin. Enzymes are selected so as to digest each represented component of the biomass. It is understood, however, that the biomass identified at 24 is representative only for the purpose of description herein.

Once a selected volume of the biomass is placed in the bioreactor (an exemplary bioreactor is described below), a selected volume of enzyme coated microbeads prepared as described above and suspended in a buffer solution are added to the bioreactor. In the embodiment of FIG. 5, the first enzyme coated microbeads are mixed cellulase coated microbeads, which preferably include a combination or mixture of endo- and exo-enzymes necessary to accomplish the desired level of biomass digestion. Once added to the bioreactor, the cellulase on the coated microbeads are allowed to digest the available cellulose in the biomass at 26. The digestion of the available cellulose to glucose is allowed sufficient time and/or subjected to advantageous environmental conditions (temperature, pH, etc.) so that the digestion of available cellulose to glucose proceeds to completion at 28. If the enzyme hydrolysis is incomplete, heat and pressure treatment in the bioreactor will be considered. The bioreactor may be constructed of a corrosion-resistant alloy for hydrothermolysis pretreatments. It is anticipated that the range of optimum reaction temperatures is 32° to 50°, with 45 C to 50 C preferred, making this process an excellent candidate for continued experiments with an SSF reaction with thermal tolerance organisms that can ferment xylose, xylobiose, glucose, and cellobiose to ethanol, butanol or hexanol.

When needed for digestion, enzymes may be returned through a line leading to the reactor chamber. Each of these digestive processes may be selectively repeated until biomass digestion is complete.

Once the digestion of cellulose to glucose is completed at 28, the bioreactor is flushed through the glucose product line exit tube. The paramagnetic cellulase coated microbeads are magnetically collected from the effluent stream and deposited in a storage container. The product feed stream is then transported for further processing, such as by fermentation into ethanol.

The enzyme (cellulase) coated microbeads recovered from the effluent stream are collected in a storage vessel into which a buffer solution is provided so as to wash the remaining biomass therefrom as described above. These cellulase coated microbeads are then suspended in a buffer solution and stored for reuse.

Following the completion of the digestion of the available cellulose to glucose in the biomass is completed and the bioreactor flushed at 30, the bioreactor containing biomass is prepared to receive the next enzyme. In FIG. 5, xylanase (mixed endo- and exo-enzymes) coated microbeads prepared as described above are introduced into the bioreactor. The xylanase is allowed to digest the available hemicellulose (xylan) component of the biomass at 32. Digestion of available hemi-celloluse (xylan) to xylose is allowed to complete under favorable time and environmental conditions at 34. Once this second enzyme hydrolysis reaction is completed, the bioreactor is flushed through a xylose product line exit tube. The xylose product line feed stream is then transported for further processing. The xylanase coated microbeads are magnetically collected from the xylose product feed stream at 36. The magnetically collected xylanase coated microbeads are placed in a holding chamber, washed with buffer solution, and suspended in buffer solution for reuse.

The bioreactor 22 is next prepared for a third hydrolysis biomass reaction, this time for the purpose of digesting the available lignin using mixed endo- and exo-ligninase/lacasse enzymes necessary to accomplish the desired level of biomass digestion. To accomplish this result, mixed ligninase/lacasse coated microbeads prepared according to the present disclosure are introduced into the bioreactor containing the biomass. The mixed ligninase/lacasse enzymes are allowed to digest the available lignin contained in the biomass to phenolic under favorable time and conditions at 38. The digestion of the available lignin in the biomass to phenolic is allowed to complete at 40. Once completed, the bioreactor is flushed through the phenolic product line exit tube at 50. The ligninase/lacasse coated microbeads are magnetically collected from the phenolic product line feed stream and deposited in an enzyme holding chamber. The phenolic product line feed stream is then transported for further processing. The collected ligninase/lacasse coated microbeads are then flushed with buffer solution so as to remove any remaining biomass and suspended in buffer solution for future reuse.

Once the digestion process of the available lignin is complete, the bioreactor is prepared for a fourth enzyme hydrolysis reaction, this time to digest the available pectin contained in the biomass using mixed endo- and exo-pectinase/pectin-lyase enzymes necessary to accomplish the desired level of biomass digestion. Pectinase/pectin-lyase enzyme coated microbeads are introduced into the bioreactor so as to digest the available the available pectin at 52. The pectin hydrolysis reaction is allowed to proceed to completion under favorable conditions so as to digest the available pectin to pectin digestive products at 54. Following digestion, the bioreactor is flushed through the pectin digestive product line exit tube at 56. The pectinase/pectin-lyase coated microbeads are magnetically removed from the pectin digestive product feed stream and collected in a holding chamber. The pectin digestive product feed stream is then transported for further processing. The pectinase/pectin-lyase coated microbeads are then washed with a buffer solution and stored in suspension in a holding chamber for reuse.

The bioreactor is then flushed of any remaining undigested biomass and recharged with new biomass containing cellulose, hemicellulose, lignin, and pectin and the entire process repeated. It should be understood, however, that the order of digestion described above may be altered as desired. In addition, it is possible that the biomass in the bioreactor could be subject to multiple digestion processes using the same enzyme through reuse of the respective enzyme coated microbead as disclosed herein.

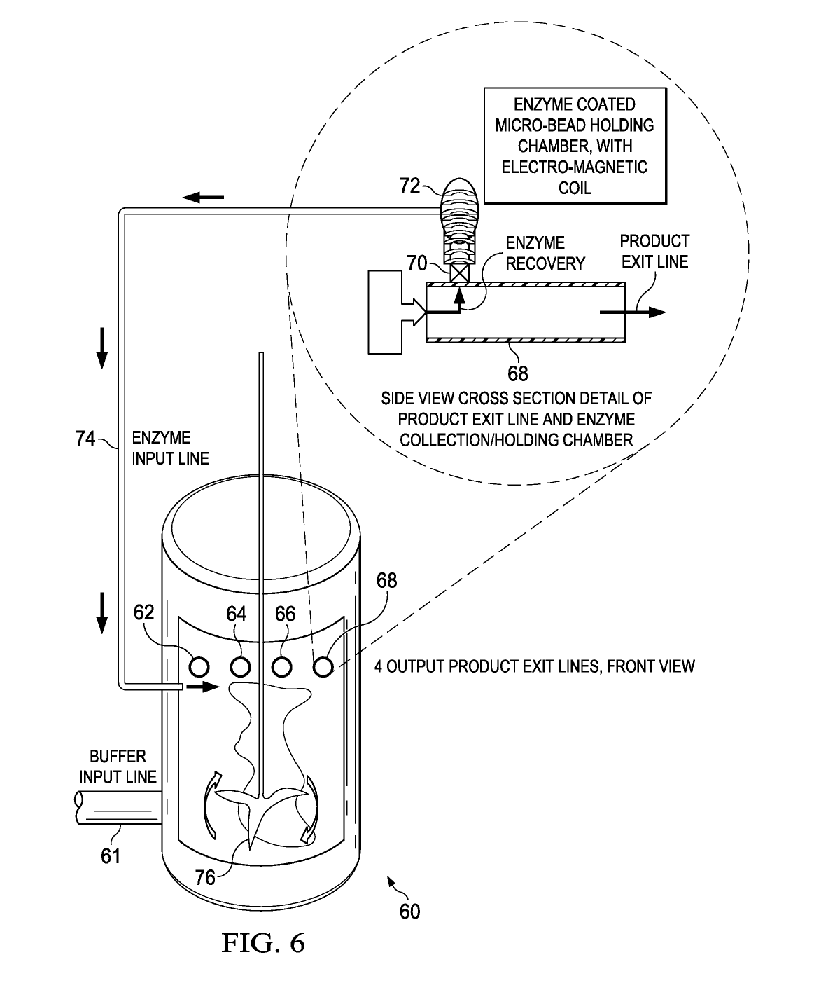

In a preferred arrangement, depicted in FIG. 6, a bioreactor 60 suitable for the embodiment of FIG. 5 is provided with a buffer input line 61 and four separate product exit (effluent) lines 62, 64, 66, and 68, one to accommodate each product type. In the embodiment of FIG. 5, four such product types are used which would require four separate product exit lines 62, 64, 66, and 68. Each of the four product lines split immediately after leaving the reactor. For example, primary product line 68 carries the product feed stream (such as glucose) including the paramagnetic particles. The paramagnetic enzyme coated particles are electro-magnetically collected through the secondary line 70 as the reaction chamber 60 is being flushed through product effluent line 68. An electro-magnetic coil surrounds an enzyme holding chamber 72 such that when reaction chamber 60 is flushed through product effluent line 68, the electromagnet is energized so as to attract the paramagnetic particles into holding chamber 72. The paramagnetic enzyme coated particles are then held in enzyme holding chamber 72 for re-injection into reaction chamber 60 through enzyme input line 74. Each product exit line 64, 66, and 68 is configured as described with reference to product exit line 62 with a separate enzyme specific holding chamber.

Thus, the present invention is well adapted to carry out the objectives and attain the ends and advantages mentioned above as well as those inherent therein. While presently preferred embodiments have been described for purposes of this disclosure, numerous changes and modifications will be apparent to those of ordinary skill in the art. Such changes and modifications are encompassed within the spirit of this invention.

What is claimed:

1. A method of recovering enzymes from enzymatic biomass hydrolysis comprising:
obtaining functionalized particles having paramagnetic properties;
attaching biomass degrading enzymes to said functionalized particles;
mixing said particles having attached enzymes with a biomass substrate to form a mixture;
recovering said particles with attached enzymes from said mixture via magnetic separation.

2. The method according to claim 1 further comprising the step of:
testing enzymatic activity retention of said functionalized particles after said step of recovery.

3. The method according to claim 1 wherein:
said enzymes are selected from a group consisting of xylonite, cellulose, lacasse, lignose, pectin-lyase and pectinase.

4. The method according to claim 1 wherein:
said functionalized particles possess nanoparticle functionalized attachment groups that are used for covalent enzyme attachment.

5. The method according to claim 4 wherein said functionalized attachment groups are selected from a group consisting of epoxide, carboxy, amine, aldehyde, DADPA, or hydrazide.

6. The method according claim 1 wherein said particles are microbeads.

7. The method according to claim 6 wherein said microbeads have a diameter of approximately 1 μm.

8. The method according to claim 1 wherein said particles are glass-iron composite spherical beads.

9. The method according to claim 1 wherein said particles are paramagnetic silica beads.

10. The method according to claim 1 wherein:
said step of recovering said particles with attached enzymes from said mixture via magnetic separation is performed on a reaction buffer.

11. The method according to claim 10 wherein:
said reaction buffer is fermentation culture effluent.

12. The method according to claim 10 wherein:
said reaction buffer is from a hydrolysis reactor.

13. The method according to claim 1 wherein:
said biomass substrate is comprised of cellulose and hemicellulose.

14. The method according to claim 13 wherein:
said biomass substrate is post harvest sorghum.

15. The method according to claim 13 wherein:
said biomass substrate is corn stover substrate.

16. The method according to claim 1 wherein:
the recovered particles with attached enzymes are suspended in a buffer solution to remove reacted biomass substrate.

17. The method according to claim 16 wherein:
the recovered particles with attached enzymes are suspended in buffer solution a plurality of times so as to produce washed recovered particles with attached enzymes.

18. The method according to claim 17 wherein:
the washed recovered particles with attached enzymes are suspended in a reaction buffer.
